(19) United States
(12) Patent Application Publication
(10) Pub. No.: US 2026/0346395 A1
(43) Pub. Date: Nov. 5, 2026

(54) POLISHING METHOD

(71) Applicants: XEBEC TECHNOLOGY CO., LTD., Tokyo (JP); TAIMEI CHEMICALS CO., LTD., Kamiina-gun, Nagano (JP)

(72) Inventor: Keisuke FUKUSHIMA, Tokyo (JP)

(21) Appl. No.: 19/107,957

(22) PCT Filed: Sep. 6, 2022
(86) PCT No.: PCT/JP2022/033388

§ 371 Date: Dec. 11, 2025

(51) Int. Cl. B24B 29/04; B24B 29/00

(52) U.S. Cl. CPC B24B 29/04; B24B 29/005

(57) ABSTRACT

A workpiece (1) is rotationally symmetrical around an axis (L0), in which a cross section of a surface (5) to be polished cut through a virtual plane including the axis (L0) has an arc (5a) exceeding 120° from a point intersecting the axis (L0) toward one side of the axis (L0). When the workpiece (1) is polished with a rotary brush (10), the workpiece (1) is rotated around the axis (L0) and the rotary brush (10) is rotated and pressed against the surface (5) to be polished, and is moved along the arc (5a) while a rotational axis (L1) of the rotary brush (10) is kept perpendicular to a tangent surface of the surface (5) to be polished. When the surface (5) to be polished and the rotary brush (10) are viewed from a rotational axis (L1) direction along the rotational axis (L1), a direction of rotation (R2) of the rotary brush (10) is opposite to a direction of rotation (R1) of the workpiece (1) on a front side of a direction of movement (M1) of the rotary brush (10).

### FIELD

The present invention relates to a polishing method for polishing a spherical part of a workpiece using a rotary brush.

### BACKGROUND

A polishing method for polishing a surface of a rotationally symmetrical workpiece with a polishing tool is described in Patent Literature 1. In this literature, the workpiece is a lens, and a surface to be polished of the workpiece is a curved surface protruding toward one side in the axial direction along the central axis. The polishing tool has an annular working section with abrasive grains embedded in an elastic material. The elastic material is a rubber, a resin, or a sponge. When the surface to be polished is polished, the workpiece is rotated around the central axis. While the polishing tool is rotated around the central axis of the working section, the working section is pressed against the surface to be polished and moved along the surface to be polished.

As a polishing tool, a rotary brush with a grinding member bundle is known. A rotary brush described in Patent Literature 2 includes: a brush body having a grinding member bundle and a grinding member bundle holder that holds a proximal end part of the grinding member bundle; and a brush case that surrounds the brush body from radially outside. The grinding member bundle is a bundle of linear grinding members made by impregnating and curing aggregate yarn of inorganic filaments such as alumina filaments with a resin. The brush case holds the brush body with a distal end part of the grinding member bundle being exposed outside.

### CITATION LIST

### Patent Literature

Patent Literature 1: Japanese Patent Application Laid-open Publication No. 2011-36974

Patent Literature 2: Japanese Patent Application Laid-open Publication No. 2009-50967

### SUMMARY

### Technical Problem

A surface of a rotationally symmetrical workpiece may be polished using the rotary brush described in Patent Literature 2. For example, a workpiece with an approximately spherical surface to be polished may be polished using a rotary brush. However, no such polishing method has been developed.

In view of the above problem, no polishing method for polishing a spherical part of a workpiece with a rotary brush has been developed.

### Solution to Problem

In order to solve the above problem, the present invention provides a polishing method for polishing a workpiece. The workpiece is rotationally symmetrical around a predetermined axis and having a surface to be polished, in which a cross section of the surface to be polished cut through a virtual plane including the axis has an arc exceeding 120° from a reference point intersecting the axis toward one side of the axis. A rotary brush is used as a polishing tool. The workpiece is rotated around the axis. The rotary brush is rotated and pressed against the surface to be polished, and is moved along the arc of the cross section while a rotational axis of the rotary brush is kept perpendicular to a tangent surface of the surface to be polished. When the surface to be polished and the rotary brush are viewed from a rotational axis direction along the rotational axis, a direction of rotation of the rotary brush is opposite to a direction of rotation of the workpiece on a front side of a direction of movement of the rotary brush.

In the present invention, the workpiece and the rotary brush are rotated when the surface to be polished of the workpiece is polished. The rotary brush is pressed against the surface to be polished and moved along the arc of the cross section while the rotational axis of the rotary brush is kept perpendicular to a tangent surface of the surface to be polished. Here, the direction of rotation of the workpiece and the direction of rotation of the rotary brush face each other on the front side of the direction of movement of the rotary brush when the surface to be polished and the rotary brush are viewed from the rotational axis direction along the rotational axis. With this configuration, the brush bites the surface to be polished, compared with when the direction of rotation of the workpiece and the direction of rotation of the rotary brush are the same direction on the front side of the direction of movement of the rotary brush. Thus, it is easier to polish the spherical surface to be polished.

In the present invention, the rotary brush may be rotated in a first direction of rotation and pressed against the surface to be polished, and may be moved along the arc of the cross section from a position offset on another side of the axis relative to the reference point to a vertex 90° away from the reference point toward one side of the axis via the reference point. The rotary brush then may be moved away from the surface to be polished. The rotary brush thereafter may be rotated in a second direction of rotation opposite to the first direction of rotation and pressed against the surface to be polished, and may be moved from a terminal end opposite to the reference point to the vertex in the arc of the cross section.

Here, when the surface to be polished of the workpiece is formed by a cutting process of cutting the workpiece by bringing a cutting tool into contact with an outer peripheral surface of the workpiece rotating around the axis, the sphericity of the part to be polished may decrease beyond a preset tolerance range due to the accuracy of the cutting process. For example, the cross section of the surface to be polished may have an oval shape that is longer in the direction orthogonal to the axis than in the axial direction. In this case, when the rotary brush is moved from the reference point toward the terminal end of the arc of the cross section of the surface to be polished, the depth of cut on the front side of the direction of movement in the rotary brush is reduced compared with the depth of cut on the rear side of the direction of movement in the rotary brush at a point in time when the rotary brush goes beyond the vertex. As a result, when the rotary brush polishes the terminal end side with respect to the vertex in the arc of the cross section, the polishing power on the surface to be polished by the rotary brush may be reduced. To address such a problem, in the present invention, when the rotary brush polishes the terminal end side with respect to the vertex in the arc of the cross section, the direction of rotation of the rotary brush is set to the second direction of rotation opposite to the first direction of rotation.

Further, the direction of movement of the rotary brush is set to the direction from the terminal end toward the vertex, and the direction of rotation of the workpiece and the direction of rotation of the rotary brush are set to directions facing each other on the front side of the direction of movement of the rotary brush. This setting can avoid the reduction of the depth of cut on the front side of the direction of movement in the rotary brush, compared with the depth of cut on the rear side of the direction of movement in the rotary brush, thereby avoiding the reduction in polishing power on the surface to be polished by the rotary brush. As a result, even when the cross section of the surface to be polished has an oval shape that is longer in a direction orthogonal to the axis, the polishing accuracy can be maintained in the same manner as when the cross section of the surface to be polished is circular.

In the present invention, the rotary brush may be rotated in a first direction of rotation and pressed against the surface to be polished, and may be moved along the arc of the cross section from a vertex 90°away from the reference point toward one side of the axis to a position offset on another side of the axis relative to the reference point via the reference point. The rotary brush then may be moved away from the surface to be polished.

The rotary brush thereafter may be rotated in a second direction of rotation opposite to the first direction of rotation and pressed against the surface to be polished, and may be moved from the vertex to a terminal end opposite to the reference point in the arc of the cross section.

Here, when the surface to be polished of the workpiece is formed by a cutting process of cutting the workpiece by bringing a cutting tool into contact with an outer peripheral surface of the workpiece rotating around the axis, the cross section of the surface to be polished may have an oval shape that is longer in the axial direction than in the direction orthogonal to the axis. In this case, when the rotary brush is moved from the reference point toward the terminal end of the arc, the depth of cut on the front side of the direction of movement in the rotary brush is reduced from the reference point toward the vertex, compared with the depth of cut on the rear side of the direction of movement in the rotary brush. As a result, when the rotary brush polishes the reference point side with respect to the vertex in the arc of the cross section of the surface to be polished, the polishing power on the surface to be polished by the rotary brush may be reduced. To address such a problem, in the present invention, when the rotary brush polishes the reference point side with respect to the vertex in the arc of the cross section, the direction of rotation of the rotary brush is set to the first direction of rotation, and the direction of movement of the rotary brush is set to a direction from the vertex toward the reference point. When the rotary brush polishes the terminal end side with respect to the vertex in the arc of the cross section, the direction of rotation of the rotary brush is set to the second direction of rotation, and the direction of movement of the rotary brush is set to a direction from the vertex toward the terminal end. This setting can avoid the reduction of the depth of cut on the front side of the direction of movement in the rotary brush, compared with the depth of cut on the rear side of the direction of movement in the rotary brush, thereby avoiding the reduction in polishing power on the surface to be polished by the rotary brush. As a result, even when the cross section of the surface to be polished has an oval shape that is longer in the axial direction, the polishing accuracy can be maintained in the same manner as when the cross section of the surface to be polished is circular.

In the present invention, the rotary brush may be rotated in the first direction of rotation and pressed against the surface to be polished, and may be moved along the arc of the cross section from a position offset on another side of the axis relative to the reference point to a terminal end opposite to the reference point in the arc of the cross section via the reference point. In other words, when the sphericity of the part to be polished is high and the cross section of the surface to be polished has a circular shape, the depth of cut on the front side of the direction of movement in the rotary brush does not change even when the rotary brush goes beyond the vertex of the arc in the cross section of the surface to be polished. As a result, the polishing power on the surface to be polished by the rotary brush can be maintained while the direction of rotation of the rotary brush is set to the first direction of rotation and the direction of movement of the rotary brush is kept in a direction from the reference point toward the terminal end.

In the present invention, it is preferable that the workpiece rotational speed at which the workpiece is rotated around the axis is lower than the brush rotational speed at which the rotary brush is rotated around the rotational axis, and the brush rotational speed is indivisible by the workpiece rotational speed. This configuration can prevent occurrence of a striped polishing pattern on the surface to be polished. In other words, when the brush rotational speed is divisible by the workpiece rotational speed, strip-shaped polishing marks occur at equal angular intervals around the axis on the surface to be polished. In contrast, when the brush rotational speed is indivisible by the workpiece rotational speed, the occurrence of such strip-shaped polishing marks can be avoided.

In the present invention, the rotary brush may include one grinding member bundle of a plurality of linear grinding members, and a grinding member bundle holder that holds a proximal end part of the grinding member bundle.

The grinding member bundle may be depressed at a central part of a distal end surface of the grinding member bundle. A distal end part of the grinding member bundle may be brought into contact with the surface to be polished. This configuration makes it easier to bring the entire distal end surface of the grinding member bundle into contact with the surface to be polished, thereby ensuring the polishing power on the surface to be polished by the rotary brush.

In the present invention, the rotary brush may include a plurality of grinding member bundles annularly arranged, and a grinding member bundle holder that holds respective proximal end parts of the grinding member bundles. Each of the grinding member bundles may include a plurality of linear grinding members. Respective distal end parts of the grinding member bundles may be brought into contact with the surface to be polished. In such a rotary brush, there is no grinding member bundle at the center of rotation where the peripheral speed is zero during polishing. Here, if the linear grinding members of the grinding member bundle come into contact with the surface to be polished of the workpiece at a peripheral speed of zero with the brush being pressed against the surface to be polished, the linear grinding members are likely to break. However, occurrence of such a situation can be avoided if the rotary brush includes a plurality of grinding member bundles annularly arranged.

In the present invention, the rotary brush may include an annular grinding member bundle of a plurality of linear grinding members annularly arranged, and a grinding member bundle holder that holds a proximal end part of the grinding member bundle. A distal end part of the grinding member bundle may be brought into contact with the surface to be polished. In such a rotary brush, there is no linear grinding member at the center of rotation of the brush where the peripheral speed is zero during polishing. Here, if the linear grinding members at a peripheral speed of zero come into contact with the surface to be polished of the workpiece with the brush being pressed against the surface to be polished, the linear grinding members are likely to break. However, occurrence of such a situation can be avoided if the rotary brush includes a grinding member bundle of a plurality of linear grinding members annularly arranged.

In the present invention, the rotary brush may include: a brush body having a grinding member bundle of a plurality of linear grinding members and a grinding member bundle holder that holds a proximal end part of the grinding member bundle; and a sleeve surrounding the grinding member bundle and the grinding member bundle holder with a distal end part of the grinding member bundle being exposed outside, the sleeve facing the grinding member bundle with a predetermined gap in a radial direction. The distal end part of the grinding member bundle may be brought into contact with the surface to be polished. With this configuration, the sleeve can restrain the grinding member bundle from spreading to the outer peripheral side, thereby ensuring the polishing power on the surface to be polished by the rotary brush. In addition, since the linear grinding members of the grinding member bundle can be restrained from going out of control, the formation of scratches on the surface to be polished of the workpiece can be prevented or suppressed.

In the present invention, the workpiece may be an artificial joint. The rotary brush may include a grinding member bundle of a plurality of linear grinding members.

The linear grinding members may each include aggregate yarn of inorganic filaments and a resin that impregnates the aggregate yarn. The rotary brush with such a bundle of linear grinding members has high grinding power. Thus, such a rotary brush can be used to polish an artificial joint made of cobalt, chrome, titanium alloy, or the like.

### BRIEF DESCRIPTION OF DRAWINGS

FIG. 1 is a side view of a workpiece to be subjected to a polishing method of the present invention.

FIG. 2 is a perspective view of a rotary brush for use in polishing a workpiece.

FIG. 3 is a cross-sectional view of the rotary brush.

FIG. 4 is a diagram illustrating a first polishing method.

FIG. 5 is a diagram illustrating a direction of rotation of the workpiece, a direction of rotation of the rotary brush, and a direction of movement of the rotary brush in the first polishing method.

FIG. 6 is a diagram illustrating a second polishing method.

FIG. 7 is a diagram illustrating a direction of rotation of the workpiece, a direction of rotation of the rotary brush, and a direction of movement of the rotary brush in the second polishing method.

FIG. 8 is a diagram for explaining an issue that occurs when the first polishing method is employed.

FIG. 9 is a diagram illustrating a third polishing method.

FIG. 10 is a diagram illustrating a direction of rotation of the workpiece, a direction of rotation of the rotary brush, and a direction of movement of the rotary brush in the third polishing method.

FIG. 11 is a diagram for explaining an issue that occurs when the first polishing method is employed.

FIG. 12 is a diagram illustrating another example of the rotary brush.

FIG. 13 is a diagram illustrating yet another example of the rotary brush.

### DESCRIPTION OF EMBODIMENTS

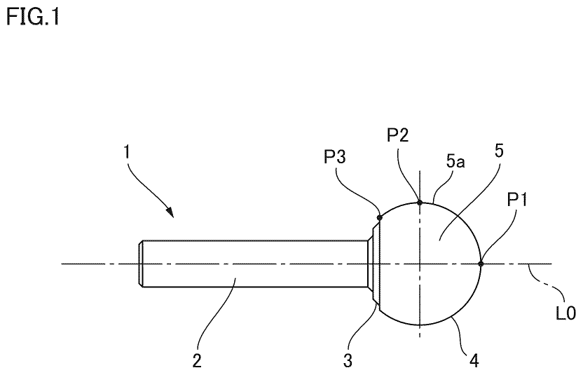

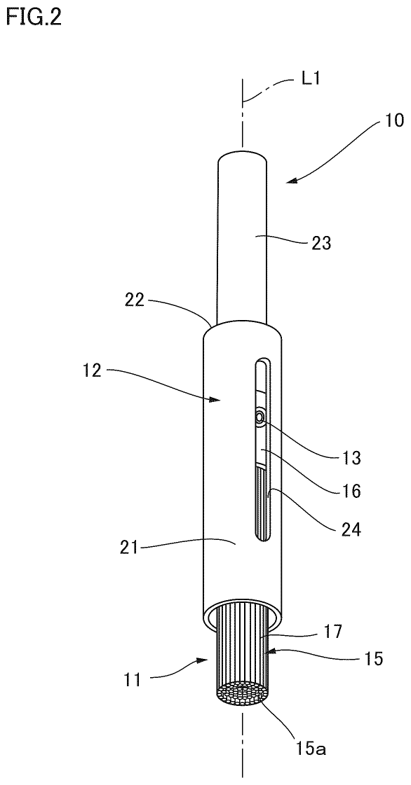

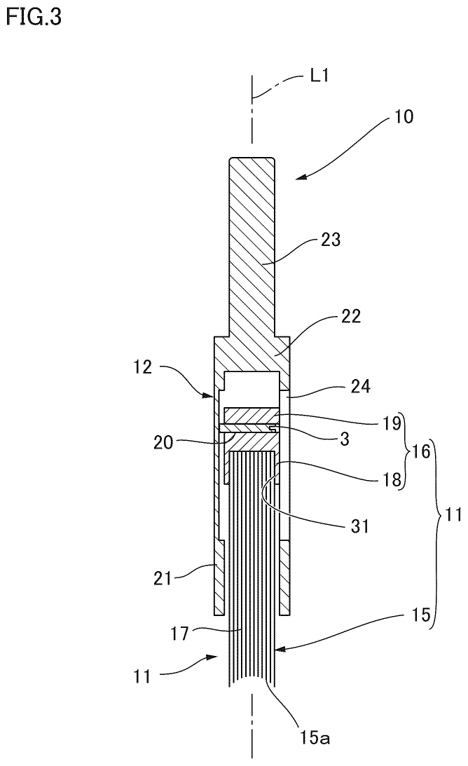

A polishing method according to embodiments of the present invention will be described below with reference to the drawings. FIG. 1 is a side view of a workpiece to be subjected to a polishing method of the present invention. FIG. 2 is a perspective view of a rotary brush for use in polishing a workpiece. FIG. 3 is a cross-sectional view of the rotary brush.

### Workpiece

A workpiece 1 to be polished by the polishing method in this example is an artificial joint. More specifically, the workpiece 1 in this example is an artificial hip joint. The workpiece 1 is made of metal, such as cobalt, chromium, or titanium alloy. As illustrated in FIG. 1, the workpiece 1 has a rotationally symmetrical shape around a predetermined axis L0. The workpiece 1 includes a rod-shaped part 2, a disk part 3, and a spherical part 4, in this order, from one side toward the other side in the axial direction X along the axis L0. A surface 5 to be polished of the workpiece 1 is an outer peripheral surface of the spherical part 4. The cross section of the surface 5 to be polished cut through a virtual plane including the axis L0 has an arc 5a exceeding 120° from a reference point P1 where the surface 5 to be polished intersects the axis L0 toward one side of the axis L0. Here, the cross section of the workpiece 1 cut through the virtual plane including the axis L0 has the same shape as the side view illustrated in FIG. 1.

In this example, the diameter of the spherical part of the workpiece 1 is from 20 mm to 40 mm. The arc 5a of the surface 5 to be polished cut through the virtual plane extends to a position at an angle 135° from the reference point P1 toward the one side of the axis L0.

When the surface 5 to be polished is polished, the workpiece is chucked at the rod-shaped part 2 onto the spindle of a machine tool.

### Rotary Brush

A polishing tool that polishes the workpiece 1 in the polishing method in this example is a rotary brush 10. As illustrated in FIG. 2 and FIG. 3, the rotary brush 10 includes a brush body 11 and a brush case 12 that holds the brush body 11. The rotary brush 10 also includes a fixing screw 13 for fixing the brush body 11 to the brush case 12.

The brush body 11 has a grinding member bundle 15 and a grinding member bundle holder 16 that holds a proximal end part of the grinding member bundle 15. The grinding member bundle 15 is a bundle of a plurality of linear grinding members 17. The linear grinding members 17 are made by impregnating and curing aggregate yarn of inorganic filaments with a binder resin. In other words, the linear grinding members 17 each include aggregate yarn of inorganic filaments such as alumina filaments and a resin that impregnates the aggregate yarn. The inorganic filaments are, for example, alumina filaments, silicon carbide fibers, boron fibers, or glass fibers.

As illustrated in FIG. 3, the grinding member bundle holder 16 includes a cylindrical portion 18 that surrounds an end part on the proximal end side of the grinding member bundle 15 from the outer peripheral side and a bottom portion 19 that closes one of the openings of the cylindrical portion 18. The bottom portion 19 is disk-shaped. The inner peripheral side of the cylindrical portion 18 and the end surface of the bottom portion 19 on the side closer to the cylindrical portion 18 constitute a grinding member bundle-holding hole 31 that holds the proximal end part of the grinding member bundle 15. An end of the grinding member bundle 15 inserted into the cylindrical portion 18 abuts on the bottom portion 19. The grinding member bundle 15 is fixed to the grinding member bundle holder 16 by adhesive injected into the grinding member bundle-holding hole 31. The grinding member bundle 15 is held in the grinding member bundle-holding hole 31 to form a circular bundle. The bottom portion 19 has a screw hole 20 that radially passes through the bottom portion 19. The screw hole 20 is used for fixing the brush body 11 to the brush case 12.

In this example, the grinding member bundle 15 is depressed at a central part of its distal end surface 15a. Thus, as illustrated in FIG. 3, when the grinding member bundle 15 is cut along a rotational axis L1 of the rotary brush 10, the distal end surface 15a has a cross section shaped like an arc 5a that is depressed toward the proximal end side of the grinding member bundle 15. The curvature of the arc 5a includes a curvature that corresponds to the curvature of the surface 5 to be polished of the workpiece 1. The diameter of the grinding member bundle 15 is preferably greater than ⅓ and equal to or less than ½ of the diameter of the spherical part 4 of the workpiece 1.

As illustrated in FIG. 2, the brush case 12 includes a sleeve 21, a circular closing portion 22 that closes a rear end of the sleeve 21, and a shank 23 that extends from the center of the closing portion 22 to the opposite side of the sleeve 21. The sleeve 21 has a slot 24 that extends in the axial direction X. The axis of the shank 23 is the rotational axis L1 of the rotary brush.

When the brush body 11 is held in the brush case 12, the brush body 11 is inserted inside the sleeve 21 so that the sleeve 21 surrounds the grinding member bundle 15 and the grinding member bundle holder 16. The distal end part of the grinding member bundle 15 is exposed outside from the sleeve 21 by a predetermined dimension. In this state, the fixing screw 13 is screwed into the screw hole 20 of the grinding member bundle holder 16 of the brush body 11 through the slot 24 of the sleeve 21.

As illustrated in FIG. 3, when the fixing screw 13 is screwed into the screw hole 20, the fixing screw 13 penetrates the grinding member bundle holder 16 in a direction orthogonal to the axis L0, and a distal end of the fixing screw 13 abuts on an opposing surface part facing the slot 24 in the inner peripheral wall surface of the sleeve 21. When the fixing screw 13 is further screwed in this state, the grinding member bundle holder 16 is pressed against a peripheral wall surface part (a peripheral wall surface part including an opening edge of the slot 24) facing the opposing surface in the inner peripheral wall surface of the sleeve 21. The brush body 11 is thus fixed to the brush case 12. When the brush body 11 is fixed to the brush case 12, the sleeve 21 faces the grinding member bundle 15 with a predetermined gap in the radial direction.

Here, the rotary brush 10 is rotated around the rotational axis L1 when polishing the workpiece 1. When the grinding member bundle 15 of the rotary brush 10 is worn due to polishing of the workpiece 1, the fixing screw 13 is loosened and the brush body 11 is moved to the opposite side of the shank 23 relative to the brush case 12 so that the distal end part of the grinding member bundle 15 is exposed from the sleeve 21 by a predetermined dimension. Thereafter, the fixing screw 13 is screwed to fix the brush body 11 to the brush case 12.

### Polishing Method

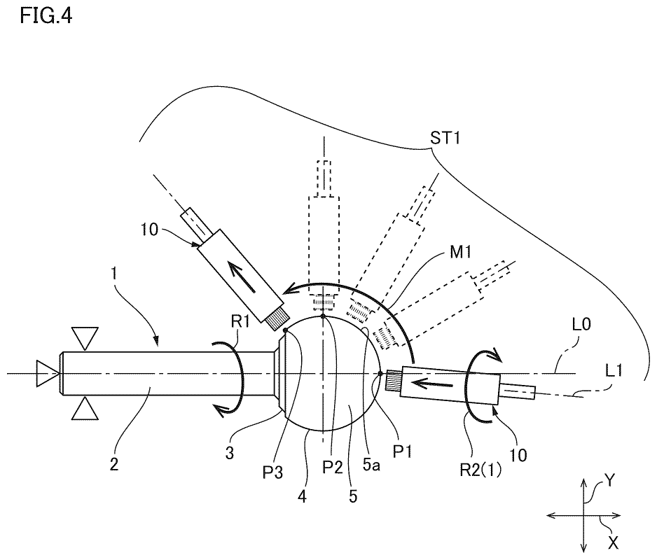

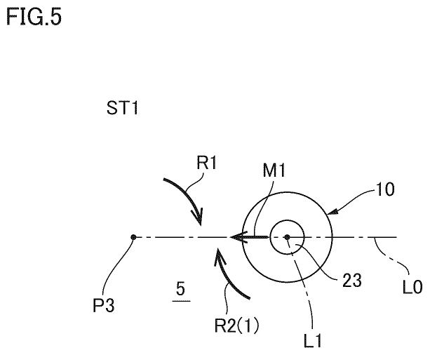

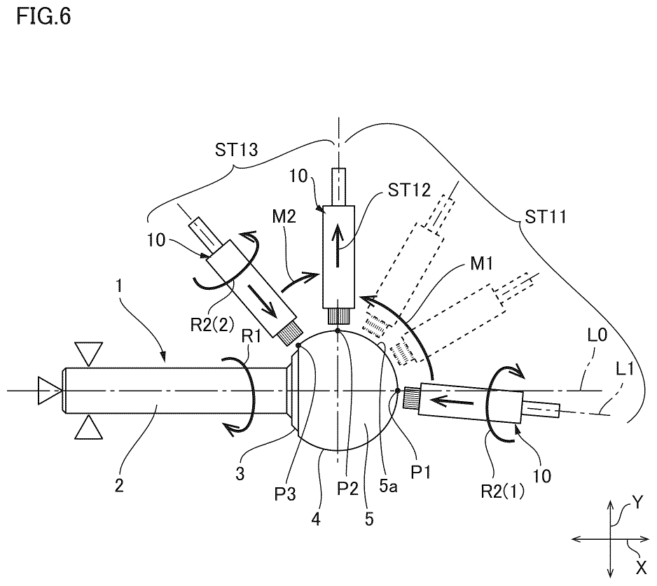

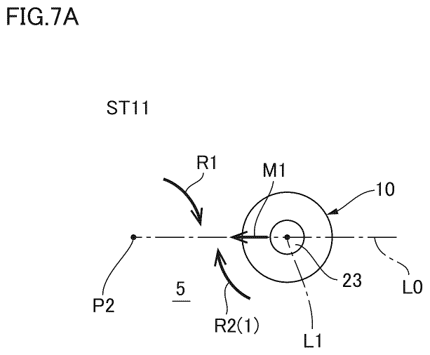

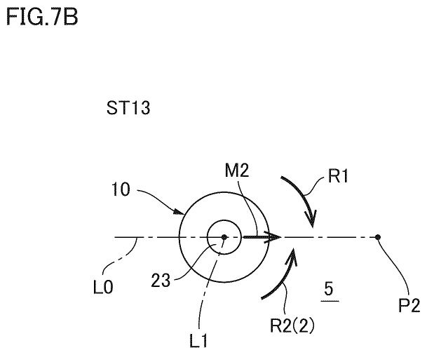

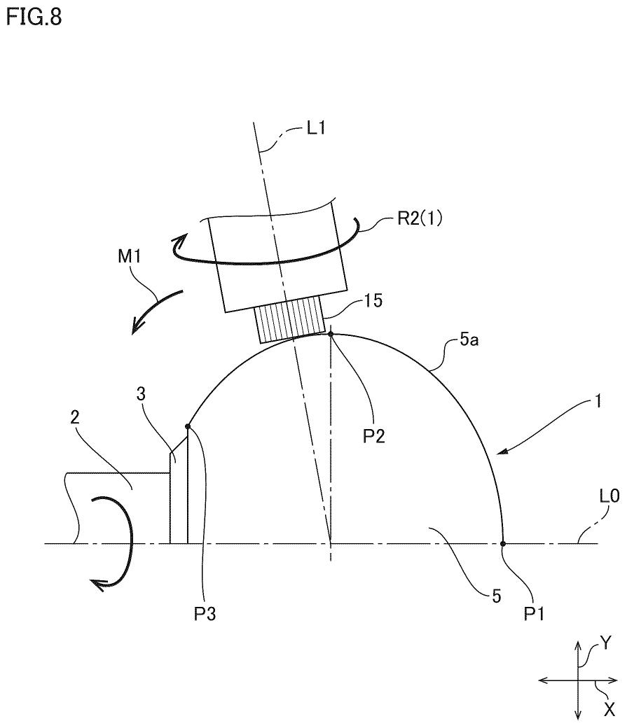

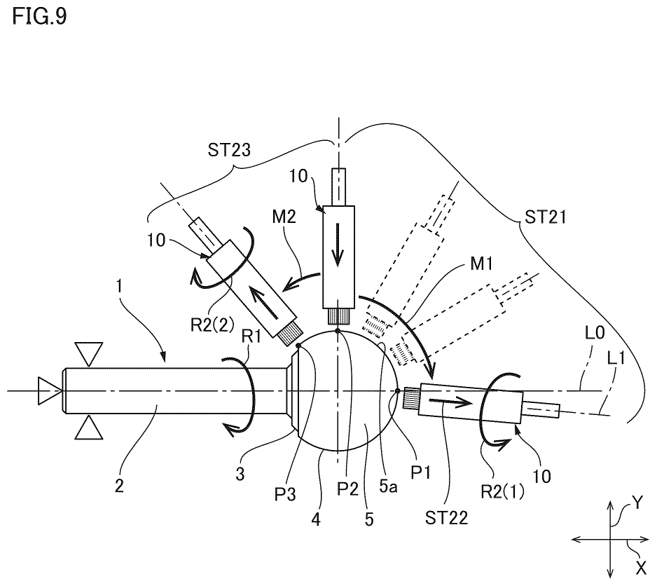

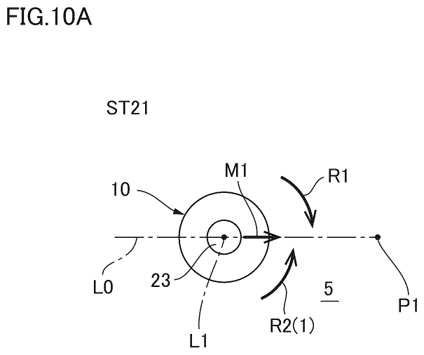

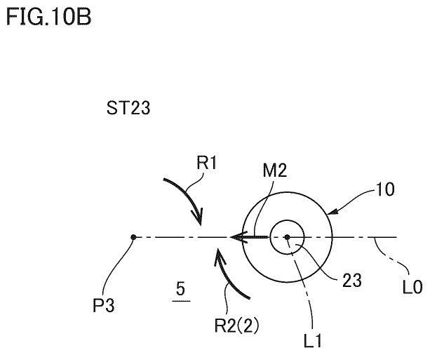

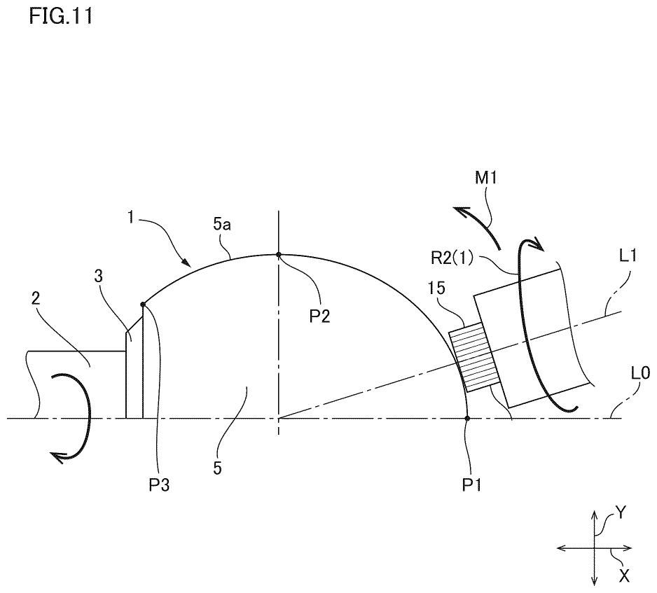

A method of polishing the workpiece 1 with the rotary brush 10 will now be described. FIG. 4 is a diagram illustrating a first polishing method. FIG. 5 is a diagram illustrating the relation between the direction of rotation of the workpiece 1, the direction of movement of the rotary brush 10, and the direction of rotation of the rotary brush 10 in the first polishing method. FIG. 6 is a diagram illustrating a second polishing method. FIG. 7 is a diagram illustrating the relation between the direction of rotation of the workpiece 1, the direction of movement of the rotary brush 10, and the direction of rotation of the rotary brush 10 in the second polishing method. FIG. 8 is a diagram for explaining an issue that occurs in the first polishing method employed when the workpiece 1 has an oval shape that is longer in a direction orthogonal to the axis L0. FIG. 9 is a diagram illustrating a third polishing method. FIG. 10 is a diagram illustrating the relation between the direction of rotation f the workpiece 1, the direction of movement of the rotary brush 10, and the direction of rotation of the rotary brush 10 in the third polishing method. FIG. 11 is a diagram for explaining an issue that occurs in the first polishing method employed when the workpiece 1 has an oval shape that is longer in the axial direction X.

As illustrated in FIG. 4 and FIG. 7, in polishing the workpiece 1, the rod-shaped part 2 of the workpiece 1 is chucked onto the spindle of a machine tool and rotated around the axis L0. The rotary brush 10 is rotated around the rotational axis L1 with the shank 23 chucked on the head of the machine tool. The rotary brush 10 is pressed against the surface 5 to be polished of the workpiece 1 by the machine tool and is moved along the arc 5a of the cross section of the surface 5 to be polished while the rotational axis L1 is kept perpendicular to a tangent surface of the surface 5 to be polished.

Here, “the rotary brush 10 is rotated and pressed against the surface 5 to be polished” means that the grinding member bundle 15 of the rotary brush 10 is brought into contact with the surface 5 to be polished with a predetermined depth of cut. A direction of rotation R1 of the workpiece 1 and a direction of rotation R2 of the rotary brush 10 face each other on the front side of a direction of movement M of the rotary brush 10 when the surface 5 to be polished and the rotary brush 10 are viewed from a rotational axis direction Y along the rotational axis L1.

A workpiece rotational speed at which the workpiece 1 is rotated around the axis L0 is lower than a brush rotational speed at which the rotary brush 10 is rotated around the rotational axis L1. The brush rotational speed and the workpiece rotational speed have such a relation that the brush rotational speed is indivisible by the workpiece rotational speed. Here, it is preferable that the speed difference between the workpiece rotational speed and the brush rotational speed is greater. A feed rate at which the rotary brush 10 is moved along the arc 5a is set based on the workpiece rotational speed and the like. The feed rate is constant.

In this example, the rotational speed of the workpiece is 300 revolutions/minute. The rotational speed of the rotary brush 10 is 10000 revolutions/min. Thus, the brush rotational speed is indivisible by the workpiece rotational speed. The feed rate of the rotary brush 10 is 0.4 mm/rev relative to the workpiece rotational speed.

Here, the polishing method for polishing the workpiece 1 using the rotary brush 10 includes: a first polishing method in which the direction of movement M1 of the rotary brush 10 and the direction of rotation R2 of the rotary brush 10 are not changed during polishing of the surface 5 to be polished; and first and second polishing methods in which the direction of movement M1 of the rotary brush 10 and the direction of rotation R2 of the rotary brush 10 are changed during polishing of the surface 5 to be polished. In all of the first polishing direction, the second polishing method, and the third polishing method, the workpiece rotational speed, the rotary brush rotational speed, and the feed rate of the rotary brush 10 are the same.

The first, the second, and the third polishing methods will be described in detail below. In the following description, a point intersecting the axis L0 on the surface 5 to be polished of the workpiece 1 is defined as reference point P1. In the arc 5a of the cross section of the surface 5 to be polished, a position 90° away from the reference point P1 toward one side of the axial direction X is defined as vertex P2 of the surface 5 to be polished. Furthermore, in the arc 5a of the cross section of the surface 5 to be polished, an end opposite to the reference point P1 is defined as terminal end P3 of the surface 5 to be polished.

### First Polishing Method

The first polishing method is used when a plurality of workpieces 1 are successively polished and the sphericity of the spherical part 4 is confirmed to be high in advance for all the workpieces 1. In other words, the first polishing method is used when the cross section of the surface 5 to be polished of the workpiece 1 has a perfect circular shape within a preset tolerance range or smaller.

As illustrated in FIG. 4, in the first polishing method, the workpiece 1 is rotated in a predetermined direction of rotation R1. The rotary brush 10 is rotated in a first direction of rotation R2(1) and pressed against the surface 5 to be polished of the rotating workpiece 1, and is moved along the arc 5a of the cross section from a position offset on the other side of the axis L0 relative to the reference point P1 to the terminal end P3 via the reference point Pl on the surface 5 to be polished of the workpiece 1 (polishing step ST1). As illustrated in FIG. 5, in the polishing step ST1, when the surface 5 to be polished and the rotary brush 10 are viewed from the rotational axis L1 direction along the rotational axis L1 of the rotary brush 10, the first direction of rotation R2(1) of the rotary brush 10 is opposite to the direction of rotation R1 of the workpiece 1 on the front side (the side where the vertex P3 is present) of the direction of movement M1 of the rotary brush 10.

Thereafter, the polishing step ST1 is repeated a predetermined number of times. The polishing of the workpiece 1 is thus completed.

### Operation Effects

In this example, when the surface 5 to be polished of the workpiece 1 is polished, the workpiece 1 and the rotary brush 10 are rotated. The rotary brush 10 is pressed against the surface 5 to be polished and moved along the arc 5a of the cross section while the rotational axis L1 of the rotary brush 10 is kept perpendicular to the tangent surface of the surface 5 to be polished. Here, the direction of rotation R1 of the workpiece 1 and the direction of rotation R2 of the rotary brush 10 face each other on the front side of the direction of movement M1 of the rotary brush 10 when the surface 5 to be polished and the rotary brush 10 are viewed from the rotational axis L1 direction along the rotational axis L1. With this configuration, the brush bites the surface 5 to be polished, compared with when the direction of rotation R1 of the workpiece 1 and the direction of rotation R2 of the rotary brush 10 are the same direction on the front side of the direction of movement M1 of the rotary brush 10. Thus, it is easier to polish the spherical surface 5 to be polished.

In this example, polishing is started from a position offset by 5° from the reference point Pl on the other side of the axis L0. Thus, the reference point P1 and the periphery thereof where the peripheral speed is zero on the surface 1 to be polished can be sufficiently polished.

The workpiece rotational speed at which the workpiece 1 is rotated around the axis L0 is lower than the brush rotational speed at which the rotary brush 10 is rotated around the rotational axis L1, and the brush rotational speed is indivisible by the workpiece rotational speed. This configuration can prevent occurrence of a striped polishing pattern on the surface 5 to be polished. In other words, when the brush rotational speed is divisible by the workpiece rotational speed, strip-shaped polishing marks occur at equal angular intervals around the axis L0 on the surface 5 to be polished. In contrast, when the brush rotational speed is indivisible by the workpiece rotational speed, the occurrence of such strip-shaped polishing marks can be avoided.

In this example, the rotary brush 10 includes one grinding member bundle 15 of a plurality of linear grinding members 17, and the grinding member bundle holder 16 that holds the proximal end part of the grinding member bundle 15. The grinding member bundle 15 is depressed at the central portion of its distal end surface 15a. The rotary brush 10 brings the distal end part of the grinding member bundle 15 into contact with the surface 5 to be polished.

This configuration makes it easier to bring the entire distal end surface 15a of the grinding member bundle 15 into contact with the surface 5 to be polished, thereby ensuring the polishing power on the surface 5 to be polished by the rotary brush 10.

The rotary brush 10 includes: the brush body 11 having the grinding member bundle 15 of a plurality of linear grinding members 17, and the grinding member bundle holder 16 that holds the proximal end part of the grinding member bundle 15; and the sleeve 21 that surrounds the grinding member bundle 15 and the grinding member bundle holder 16 with the distal end part of the grinding member bundle 15 being exposed outside and faces the grinding member bundle 15 with a predetermined gap in the radial direction. The rotary brush 10 brings the distal end part of the grinding member bundle 15 into contact with the surface 5 to be polished. According to this example, the sleeve 21 can restrain the grinding member bundle 15 from spreading to the outer peripheral side, thereby ensuring the polishing power on the surface 5 to be polished by the rotary brush 10. In addition, since the linear grinding members 17 of the grinding member bundle 15 can be restrained from going out of control, the formation of scratches on the surface 5 to be polished of the workpiece 1 can be prevented or suppressed.

The workpiece 1 is an artificial joint. The rotary brush 10 includes the grinding member bundle 15 of a plurality of linear grinding members 17. The linear grinding members 17 each include aggregate yarn of inorganic filaments and a resin that impregnates the aggregate yarn. The rotary brush 10 with such a bundle of linear grinding members 17 has high grinding power. Thus, an artificial joint made of cobalt, chromium, titanium alloys, or the like can be polished.

### Second Polishing Method

When the surface 5 to be polished of the workpiece 1 is formed by a cutting process of cutting the workpiece by bringing a cutting tool into contact with an outer peripheral surface of the workpiece 1 rotating around the axis L0, the sphericity of the spherical part 4 may decrease beyond a preset tolerance range due to the accuracy of the cutting process. When the sphericity decreases due to the accuracy of the cutting process, the cross section of the surface 5 to be polished of the workpiece 1 may tend to become an oval shape that is longer in the Y direction orthogonal to the axis L0 than in the axial direction X. The second polishing method is used when a plurality of workpieces 1 are to be polished and may include a workpiece 1 having such a shape.

As illustrated in FIG. 6, in the second polishing method, first, the workpiece 1 chucked on the spindle of a machine tool is rotated in the direction of rotation R1, and the rotary brush 10 chucked on the head of the machine tool is rotated in the first direction of rotation R2(1).

The rotary brush 10 is pressed against the surface 5 to be polished of the rotating workpiece 1 and moved along the arc 5a of the cross section of the surface 5 to be polished from a position offset on the other side of the axis L0 relative to the reference point P1 to the vertex P2 via the reference point P1 (first polishing step ST11). In the second polishing direction, the direction of rotation R1 of the workpiece 1 is the same as in the first polishing method.

Here, while the rotary brush 10 moves, the rotational axis L1 of the rotary brush 10 is kept perpendicular to the tangent surface of the surface 5 to be polished. As illustrated in FIG. 7A, when the surface 5 to be polished and the rotary brush 10 are viewed from the rotational axis L1 direction along the rotational axis L1 of the rotary brush 10, the second direction of rotation R2(2) of the rotary brush 10 is opposite to the direction of rotation R1 of the workpiece 1 on the front side (the side where the vertex P2 is present) of the direction of movement M1 of the rotary brush 10.

Next, the rotary brush 10 is moved away from the surface 5 to be polished (setting change step ST12).

Thereafter, the rotary brush 10 is rotated in the second direction of rotation R2(2) opposite to the first direction of rotation R2(1) and pressed against the surface 5 to be polished, and is moved from the terminal end P3 to the vertex P2 of the arc 5a (second polishing step ST13).

Thus, the direction of movement M2 of the rotary brush 10 is opposite to the direction of movement M1 in the first polishing step. The direction of rotation R1 of the workpiece 1 remains unchanged.

Here, while the rotary brush 10 moves, the rotational axis L1 of the rotary brush 10 is kept perpendicular to the tangent surface of the surface 5 to be polished. As illustrated in FIG. 7B, when the surface 5 to be polished and the rotary brush 10 are viewed from the rotational axis L1 direction along the rotational axis L1 of the rotary brush 10, the second direction of rotation R2(2) of the rotary brush 10 is opposite to the direction of rotation R1 of the workpiece 1 on the front side (the side where the vertex P2 is present) of the direction of movement M1 of the rotary brush 10.

Thereafter, the first polishing step ST11, the setting change step ST12, and the second polishing step ST13 are repeated a predetermined number of times. The polishing of the workpiece 1 is thus completed.

### Operation Effects of Second Polishing Method

In the second polishing method, even when the cross section of the surface 5 to be polished has an oval shape that is longer in the direction Y orthogonal to the axis L0 than in the axial direction X due to the processing accuracy of the workpiece 1, the polishing accuracy can be maintained in the same manner as when the cross section of the surface 5 to be polished is circular. This effect will be explained with reference to FIG. 8. In FIG. 8, the cross section of the surface 5 to be polished of the workpiece 1 has an oval shape that is longer in the direction Y orthogonal to the axis L0 than in the axial direction X.

For example, suppose that when the cross section of the surface 5 to be polished has an oval shape that is longer in the direction Y orthogonal to the axis L0, the first polishing method is employed to perform polishing by moving the rotary brush 10 from the reference point P1 toward the terminal end P3 of the arc 5a. In this case, as illustrated in FIG. 8 in an exaggerated manner, when the rotary brush 10 moving from the reference point P1 toward the terminal end P3 of the arc 5a goes beyond the vertex P2, at the point in time when the rear part in the direction of movement M1 in the grinding member bundle 15 comes into contact with the surface 5 to be polished, the front part in the direction of movement M1 in the grinding member bundle 15 fails to come into contact with the surface 5 to be polished at the same depth as the rear part. Thus, the depth of cut on the front side of the direction of movement in the rotary brush 10 is reduced, compared with the depth of cut on the rear side of the direction of movement in the rotary brush 10. As a result, when the rotary brush 10 polishes the terminal end P3 side with respect to the vertex P2 in the arc 5a of the cross section, the polishing power on the surface 5 to be polished by the rotary brush 10 may be reduced.

In contrast, in this example, when the rotary brush 10 polishes the terminal end P3 side with respect to the vertex P2 in the arc 5a of the cross section, the direction of rotation R2 of the rotary brush 10 is set to the second direction of rotation R2(2) opposite to the first direction of rotation R2(1). Further, the direction of movement M1 of the rotary brush 10 is set to the direction from the terminal end P3 toward the vertex P2, and the direction of rotation R1 of the workpiece 1 and the direction of rotation R2 of the rotary brush 10 are set to directions facing each other on the front side of the direction of movement M1 of the rotary brush 10. This setting can avoid the reduction of the depth of cut on the front side of the direction of movement in the rotary brush 10, compared with the depth of cut on the rear side of the direction of movement in the rotary brush 10, thereby avoiding the reduction in polishing power on the surface 5 to be polished by the rotary brush 10. As a result, the polishing accuracy can be maintained even when the cross section of the surface 5 to be polished has an oval shape.

The second polishing method can achieve operation effects similar to those of the first polishing method.

### Third Polishing Method

When the surface 5 to be polished of the workpiece 1 is formed by a cutting process of cutting the workpiece by bringing a cutting tool into contact with an outer peripheral surface of the workpiece 1 rotating around the axis L0, the sphericity of the spherical part 4 may decrease beyond a preset tolerance range due to the accuracy of the cutting process. When the sphericity decreases due to the accuracy of the cutting process, the cross section of the surface 5 to be polished of the workpiece 1 may tend to become an oval shape that is longer in the axial direction X than in the Y direction orthogonal to the axis L0. The third polishing method is used when a plurality of workpieces 1 are to be polished and may include a workpiece 1 having such a shape.

As illustrated in FIG. 9, in the third polishing method, first, the workpiece 1 chucked on the spindle of a machine tool is rotated in the direction of rotation R1, and the rotary brush 10 chucked on the head of the machine tool is rotated in the first direction of rotation R2(1).

The rotary brush 10 is pressed against the surface 5 to be polished of the rotating workpiece 1 and moved along the arc 5a of the cross section of the surface 5 to be polished from the vertex P2 to a position offset on the other side of the axis L0 relative to the reference point P1 via the reference point P1 (first polishing step ST21). In the third polishing direction, the direction of rotation R1 of the workpiece 1 is the same as in the first and second polishing methods. The first direction of rotation R2(1) in the third polishing direction is the direction of rotation opposite to the first direction of rotation R2(1) in the second polishing method. The direction of movement M1 of the rotary brush 10 in the third polishing direction is the direction opposite to the direction of movement M1 of the rotary brush 10 in the third polishing direction.

Here, while the rotary brush 10 moves, the rotational axis L1 of the rotary brush 10 is kept perpendicular to the tangent surface of the surface 5 to be polished. As illustrated in FIG. 10A, when the surface 5 to be polished and the rotary brush 10 are viewed from the rotational axis L1 direction along the rotational axis L1 of the rotary brush 10, the first direction of rotation R2(1) of the rotary brush 101 is opposite to the direction of rotation R1 of the workpiece 1 on the front side (the side where the reference point P1 is present) of the direction of movement M1 of the rotary brush 10.

Next, the rotary brush 10 is moved away from the surface 5 to be polished (setting change step ST22).

Thereafter, the rotary brush 10 is rotated in the second direction of rotation R2(2) opposite to the first direction of rotation R2(1) and pressed against the surface 5 to be polished, and is moved from the vertex P2 to the terminal end P3 (second polishing step ST23). Here, the second direction of rotation R2(2) in the third polishing direction is the direction of rotation opposite to the second direction of rotation R2(2) in the second polishing method. The direction of movement M2 of the rotary brush 10 in the third polishing direction is the direction opposite to the direction of movement M1 of the rotary brush 10 in the third polishing direction and is the same direction as the direction of movement M1 in the first polishing method. The direction of rotation R1 of the workpiece 1 remains unchanged.

Here, while the rotary brush 10 moves, the rotational axis L1 of the rotary brush 10 is kept perpendicular to the tangent surface of the surface 5 to be polished. As illustrated in FIG. 10B, when the surface 5 to be polished and the rotary brush 10 are viewed from the rotational axis L1 direction along the rotational axis L1 of the rotary brush 10, the second direction of rotation R2(2) of the rotary brush 10 is opposite to the direction of rotation R1 of the workpiece 1 on the front side (the side where the terminal end P3 is present) of the direction of movement M1 of the rotary brush 10.

Thereafter, the first polishing step ST21, the setting change step ST22, and the second polishing step ST23 are repeated a predetermined number of times. The polishing of the workpiece 1 is thus completed.

### Operation Effects of Third Polishing Method

In the third polishing method, even when the cross section of the surface 5 to be polished has an oval shape that is longer in the axial direction X due to the processing accuracy of the workpiece 1, the polishing accuracy can be maintained in the same manner as when the cross section of the surface 5 to be polished is circular. This effect will be explained with reference to FIG. 11.

In FIG. 11, the cross section of the surface 5 to be polished of the workpiece 1 has an oval shape that is longer in the axial direction X than in the direction Y orthogonal to the axis L0.

For example, suppose that when the cross section of the surface 5 to be polished has an oval shape that is longer in the axial direction X, the first polishing method is employed to perform polishing by moving the rotary brush 10 from the reference point P1 toward the terminal end P3 of the arc 5a. In this case, as illustrated in FIG. 11 in an exaggerated manner, from the reference point P1 to the vertex P2 of the arc 5a, at the point in time when the rear part in the direction of movement M1 in the grinding member bundle 15 comes into contact with the surface 5 to be polished, the front part in the direction of movement M1 in the grinding member bundle 15 fails to come into contact with the surface 5 to be polished at the same depth as the rear part. Thus, the depth of cut on the front side of the direction of movement in the rotary brush 10 is reduced, compared with the depth of cut on the rear side of the direction of movement in the rotary brush 10. As a result, when the rotary brush 10 polishes the reference point P1 side with respect to the vertex P2, the polishing power on the surface 5 to be polished by the rotary brush 10 may be reduced.

In contrast, in this example, when the rotary brush 10 polishes the reference point P1 side with respect to the vertex P2, the direction of movement M1 of the rotary brush 10 is set to the direction from the vertex P2 toward the reference point P1. The direction of rotation R1 of the workpiece 1 and the direction of rotation R2 of the rotary brush 10 are set to directions facing each other on the front side of the direction of movement M1 of the rotary brush 10. This setting can avoid the reduction of the depth of cut on the front side of the direction of movement in the rotary brush 10, compared with the depth of cut on the rear side of the direction of movement in the rotary brush 10, thereby avoiding the reduction in polishing power on the surface 5 to be polished by the rotary brush 10. As a result, the polishing accuracy can be maintained even when the cross section of the surface 5 to be polished has an oval shape.

The third polishing method can achieve operation effects similar to those of the first polishing method.

### Modifications

The rotary brush 10 does not necessarily include the sleeve 21. For example, the brush body 11 can be used as a rotary brush.

In polishing of the workpiece 1, the rotary brush 10 may be attached to the head of a machine tool via a floating holder, so that the rotary brush 10 is pressed against the surface 5 to be polished with a predetermined biasing force.

### Another Example of Rotary Brush

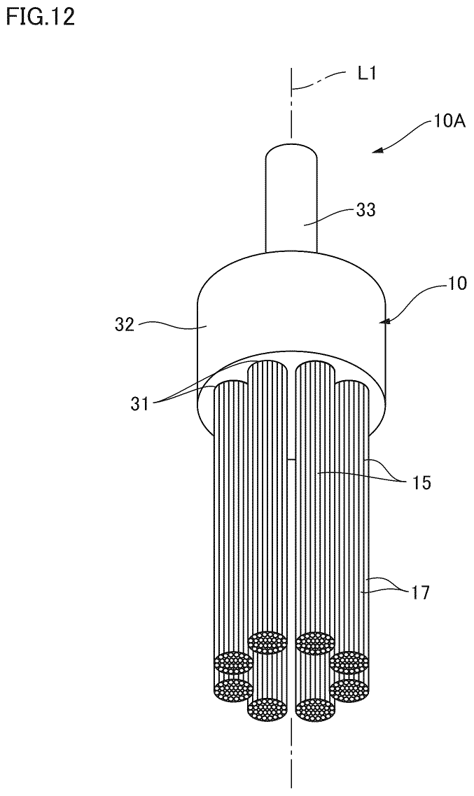

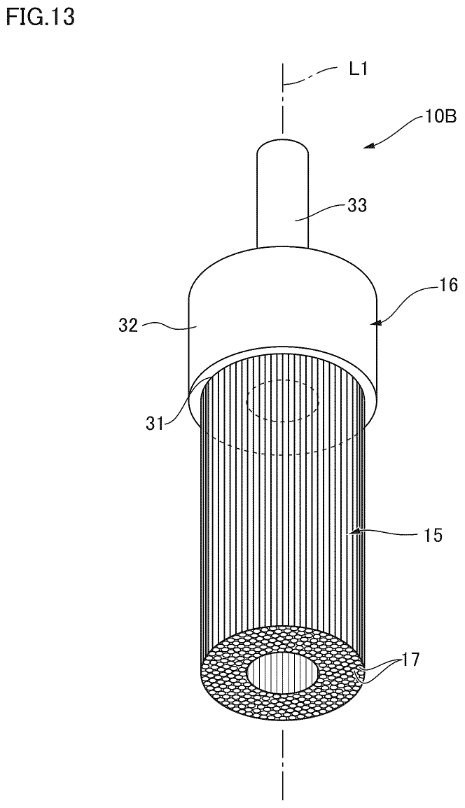

FIG. 12 is a diagram illustrating another rotary brush for use in the polishing method in this example. FIG. 13 is a diagram illustrating yet another rotary brush for use in the polishing method in this example. A rotary brush 10A illustrated in FIG. 12 includes a plurality of grinding member bundles 15 annularly arranged and a grinding member bundle holder 16 that holds respective proximal end parts of the grinding member bundles 15. Each of the grinding member bundles 15 includes a plurality of linear grinding members 17. The grinding member bundle holder 16 has a disk portion 32 with a plurality of grinding member bundle-holding holes 31 annularly arranged on its front surface, and a shank portion 33 that extends backward from the center of the disk portion 32. Each grinding member bundle 15 has a proximal end part inserted in the corresponding grinding member bundle-holding hole 31 and fixed to the grinding member bundle holder 16 by adhesive. In polishing of the workpiece 1, the respective distal end parts of the grinding member bundles 15 are brought into contact with the surface 5 to be polished.

In the rotary brush 10A in this example, there is no grinding member bundle 15 at the center of rotation where the peripheral speed is zero during polishing. Here, if the linear grinding members 17 of the grinding member bundle 15 come into contact with the surface 5 to be polished of the workpiece 1 at a peripheral speed of zero with the brush being pressed against the surface 5 to be polished, the linear grinding members 17 are likely to break. However, occurrence of such a situation can be avoided if the rotary brush 10A includes a plurality of grinding member bundles 15 annularly arranged. The rotary brush 10A may also include a sleeve 21 that surrounds a plurality of grinding member bundles 15 and the grinding member bundle holder 16 with a distal end part of each grinding member bundle 15 being exposed outside.

A rotary brush 10B illustrated in FIG. 13 includes an annular grinding member bundle 15 of a plurality of linear grinding members 17 annularly arranged and a grinding member bundle holder 16 that holds a proximal end part of the grinding member bundle 15. The grinding member bundle holder 16 has a disk portion 32 with one annular grinding member bundle-holding hole 31 on its front surface and a shank portion 33 that extends backward from the center of the disk portion 32. The grinding member bundle 15 has the proximal end part inserted in the grinding member bundle-holding hole 31 and fixed to the grinding member bundle holder 16 by adhesive. In a polishing process, the respective distal end parts of the grinding member bundles 15 are brought into contact with the surface 5 to be polished.

In the rotary brush 10B, there is no linear grinding member 17 at the center of rotation where the peripheral speed is zero during polishing. Thus, it is possible to suppress the breakage of the linear grinding member 17 coming into contact with the surface 5 to be polished of the workpiece 1 at a peripheral speed of zero. If the rotary brush 10B has the annular grinding member bundle 15, the grinding member bundle 15 continuously comes into contact with the surface 5 to be polished. Thus, it is easier to improve the surface roughness of the surface 5 to be polished after polishing, compared with the rotary brush 10A in which a plurality of linear grinding members are annularly arranged and the grinding member bundles 15 intermittently come into contact with the surface 5 to be polished during polishing. The rotary brush 10B may also include a sleeve 21 that surrounds a plurality of grinding member bundles 15 and the grinding member bundle holder 16 with a distal end part of each grinding member bundle 15 being exposed outside.

1. A polishing method for polishing a workpiece, the workpiece being rotationally symmetrical around a predetermined axis and having a surface to be polished, in which a cross section of the surface to be polished cut through a virtual plane including the axis has an arc exceeding 120° from a reference point intersecting the axis toward one side of the axis, wherein
a rotary brush is used as a polishing tool,
the workpiece is rotated around the axis,
the rotary brush is rotated and pressed against the surface to be polished, and is moved along the arc of the cross section while a rotational axis of the rotary brush is kept perpendicular to a tangent surface of the surface to be polished, and
when the surface to be polished and the rotary brush are viewed from a rotational axis direction along the rotational axis, a direction of rotation of the rotary brush is opposite to a direction of rotation of the workpiece on a front side of a direction of movement of the rotary brush.

2. The polishing method according to claim 1, wherein
the rotary brush is rotated in a first direction of rotation and pressed against the surface to be polished, and is moved along the arc of the cross section from a position offset on another side of the axis relative to the reference point to a vertex 90° away from the reference point toward one side of the axis via the reference point,
the rotary brush is then moved away from the surface to be polished, and
the rotary brush is thereafter rotated in a second direction of rotation opposite to the first direction of rotation and pressed against the surface to be polished, and is moved from a terminal end opposite to the reference point to the vertex in the arc of the cross section.

3. The polishing method according to claim 1, wherein
the rotary brush is rotated in a first direction of rotation and pressed against the surface to be polished, and is moved along the arc of the cross section from a vertex 90° away from the reference point toward one side of the axis to a position offset on another side of the axis relative to the reference point via the reference point,
the rotary brush is then moved away from the surface to be polished, and
the rotary brush is thereafter rotated in a second direction of rotation opposite to the first direction of rotation and pressed against the surface to be polished, and is moved from the vertex to a terminal end opposite to the reference point in the arc of the cross section.

4. The polishing method according to claim 1, wherein the rotary brush is rotated in a first direction of rotation and pressed against the surface to be polished, and is moved along the arc of the cross section from a position offset on another side of the axis relative to the reference point to a terminal end opposite to the reference point in the arc of the cross section via the reference point.

5. The polishing method according to claim 1, wherein
a workpiece rotational speed at which the workpiece is rotated around the axis is lower than a brush rotational speed at which the rotary brush is rotated around the rotational axis, and
the brush rotational speed is indivisible by the workpiece rotational speed.

6. The polishing method according to claim 1, wherein
the rotary brush includes one grinding member bundle of a plurality of linear grinding members, and a grinding member bundle holder that holds a proximal end part of the grinding member bundle,
the grinding member bundle is depressed at a central part of a distal end surface of the grinding member bundle, and
a distal end part of the grinding member bundle is brought into contact with the surface to be polished.

7. The polishing method according to claim 1, wherein
the rotary brush includes a plurality of grinding member bundles annularly arranged, and a grinding member bundle holder that holds respective proximal end parts of the grinding member bundles,
each of the grinding member bundles includes a plurality of linear grinding members, and
respective distal end parts of the grinding member bundles are brought into contact with the surface to be polished.

8. The polishing method according to claim 1, wherein
the rotary brush includes an annular grinding member bundle of a plurality of linear grinding members annularly arranged, and a grinding member bundle holder that holds a proximal end part of the grinding member bundle, and
a distal end part of the grinding member bundle is brought into contact with the surface to be polished.

9. The polishing method according to claim 1, wherein
the rotary brush includes: a brush body having a grinding member bundle of a plurality of linear grinding members and a grinding member bundle holder that holds a proximal end part of the grinding member bundle; and a sleeve surrounding the grinding member bundle and the grinding member bundle holder with a distal end part of the grinding member bundle being exposed outside, the sleeve facing the grinding member bundle with a predetermined gap in a radial direction, and
the distal end part of the grinding member bundle is brought into contact with the surface to be polished.

10. The polishing method according to claim 1, wherein
the workpiece is an artificial joint,
the rotary brush includes a grinding member bundle of a plurality of linear grinding members, and
the linear grinding members each include aggregate yarn of inorganic filaments and a resin that impregnates the aggregate yarn.
